(19) United States
(12) Patent Application Publication
(10) Pub. No.: US 2026/0007807 A1
(43) Pub. Date: Jan. 8, 2026

(54) MEDICINAL PRODUCT OF HIGH LIPID EMULSION STABILITY, CONSISTING OF POLYCARBONATE MATERIAL

(71) Applicant: Covestro Deutschland AG, Leverkusen (DE)

(72) Inventors: Bernd GARSKA, Köln (DE); Chanita SUNGKAPREECHA, Koeln (DE); Kristina PUPOVAC, Düsseldorf (DE); Ulrike LUETZOW, Köln (DE)

(21) Appl. No.: 19/131,269

(22) PCT Filed: Nov. 27, 2023
(86) PCT No.: PCT/EP2023/083103

§ 371 Date: May 20, 2025

(30) Foreign Application Priority Data
Dec. 1, 2022 (EP) 22210849.0

(51) Int. Cl. A61L 29/06; A61M 5/14

(52) U.S. Cl. CPC A61L 29/06; A61M 5/14; A61M 2202/0482

(57) ABSTRACT

The present application describes the use of functionalized siloxane polymer for improving the intralipid resistance of aromatic polycarbonate, as is relevant especially for medical devices comprising elements which are intended to come into contact with intralipid solution during the intended use of the medical devices.

The present invention relates to medical devices comprising elements made of thermoplastic which are intended to come into contact with intralipid during the intended use of the medical device.

Aromatic polycarbonate is an attractive material that is also used in some fields of medical technology. However, depending on the required profile of properties suitable polycarbonate-based compositions have hitherto been identified for far from all desired fields of application. However, in the context of the circular economy it would be desirable to also be able to use polycarbonate in products other than those known to date, for example in autoinjectors, injection aids and dialyzers, in order to use as few different materials as possible and thus possibly allow them to be recycled together.

For medical devices such as three-way stopcocks for enteral or parenteral nutrition, intralipid resistance is of great importance. Intralipid is a lipid emulsion based on soybean oil. In artificial nutrition by intravenous application it serves as a fat source. It also has further advantageous properties, for example improves the bioavailability of anticancer drugs based on platinum. It also appears to support the treatment of autoimmune diseases by reducing the activity of natural killer cells and to promote treatment in case of recurring miscarriages.

The suitable material currently used for such medical devices brought into contact with intralipids during their intended use is POM (polyoxymethylene). By contrast, polycarbonate has not been an attractive material for such applications to date. The chemicals resistance of aromatic polycarbonate as such is limited and, if contact of the material with intralipids is to occur, even conventional specialty polycarbonate considered chemicals-resistant is not suitable for such applications.

Increasing the chemicals resistance of polycarbonate typically comprises increasing the molecular weight. However, this is only possible up to a certain extent, up to molecular weights M_{w} of about 31 000 to 34 000 g/mol, determined by GPC, calibrated against bisphenol A-polycarbonate standards with dichloromethane as eluent. Melt flowability and thus processibility are ever decreasing with increasing molecular weight.

The problem addressed by the present invention is accordingly that of providing an intralipid-resistant polycarbonate material that gains intralipid resistance by other means than by increasing molecular weight for corresponding use in medical devices intended for direct contact with intralipids.

It has surprisingly been found that compositions based on aromatic polycarbonate have a markedly improved intralipid resistance when they contain functionalized siloxane polymer composed of siloxane units, in particular polydimethylsiloxane. This positive influence is also evident at relatively low molecular weights of the polycarbonate which would otherwise have declassified the material as unsuitable for corresponding medical devices, i.e. polycarbonate having a melt volume flow rate MVR of 15 to 35 cm³/(10 min), determined according to ISO 1133:2012-03, at a test temperature of 300° C. and a load of 1.2 kg. Corresponding embodiments with polycarbonates having rather low molecular weights are particularly attractive since compositions which are very readily processible without further additivation for flow improvement, as would otherwise be necessary, may be employed.

The invention accordingly provides a
medical device or part of a medical device,
wherein the medical device or part of a medical device comprises an element consisting of a thermoplastic composition containing the following components:
A) at least 75% by weight of aromatic polycarbonate and
B) 0.2% to 1.5% by weight of functionalized siloxane polymer composed only of siloxane units as monomer units,
wherein the reported amounts are based on the total weight of the thermoplastic composition and wherein the element is intended to be in contact with intralipid during the intended use of the medical device.

The invention further relates to the use of functionalized siloxane polymer composed only of siloxane units as monomer units for improving the intralipid resistance of compositions based on aromatic polycarbonate.

It will be appreciated that a “medical device comprising an element consisting of a defined composition” or “part of a medical device comprising an element consisting of a defined composition” also includes medical devices or parts of medical devices consisting “only” of the defined composition. It will likewise be appreciated that the “element” is especially a molded part made of the thermoplastic composition and that “consisting of” does not preclude further layers or connecting elements being attached to the element. It is conceivable for the element consisting of the thermoplastic composition to comprise for example one or more coating layers which, however, do not extend over its entire surface. The “element” may also be merely a subregion of a molded part obtained for instance by multicomponent injection molding, wherein precisely this element, this subregion is brought into direct contact with intralipid when the medical device is used for its intended purpose. The element may likewise be a coating layer or a coextruded layer of a molded part made of another thermoplastic material which is present at least in the region in which the molded part is in contact with intralipid during its intended use.

For reasons of cost saving and since the thermoplastic composition used according to the invention already has a very high intralipid resistance, the molded part made of the thermoplastic composition preferably has no further layers.

Intralipid which during the use of the medical device as intended is in immediate, i.e. direct, contact with the element made of the thermoplastic composition is preferably of a type intended for parenteral nutrition, preferably of a type containing medium-chain triglycerides, wherein “medium-chain” is preferably to be understood as meaning C₆ to C₁₂. Corresponding intralipids typically contain a mixture of soybean oil, glycerol and phospholipids from eggs. Such a typical intralipid is for example a mixture containing amino acids such as alanine, arginine, glycine, histidine, isoleucine, leucine, lysine acetate, methionine, phenylalanine, proline, serine, taurine, threonine, tryptophan, tyrosine, valine, but also refined soybean oil, medium-chain triglycerides, refined olive oil, omega-3 acid-rich fish oil, glucose (carbohydrates), nitrogen, acetate (content from amino acid solution), phosphate (content from lipid emulsion), glycerol, egg lecithin, alpha-tocopherol (Ph.Eur.), sodium hydroxide, sodium oleate, acetic acid 99%, hydrochloric acid 10%, water. The intralipid is preferably SmofKabiven electrolyte-free emulsion from Fresenius Kabi AG.

It is preferable when the medical device or part of a medical device is a tube connector, a three-way stopcock, a Luer connector, a manifold, a drip chamber, an IV catheter.

### Component A

Component A of the compositions according to the invention are aromatic polycarbonates.

Aromatic polycarbonates in the context of the present invention include not only homopolycarbonates but also copolycarbonates and/or polyester carbonates; the polycarbonates may be linear or branched in a known manner. It is also possible according to the invention to employ mixtures of polycarbonates.

The melt volume flow rate MVR of the employed aromatic polycarbonate, determined according to ISO 1133:2012-03, at a test temperature of 300° C. and a load of 1.2 kg, is preferably 6 to 35 cm³/(10 min), more preferably 15 cm³/(10 min) to 35 cm³/(10 min), yet more preferably 16 to 30 cm³/(10 min), particularly preferably 16 to 20 cm³/(10 min), very particularly preferably 18 to 20 cm³/(10 min). If the thermoplastic compositions used according to the invention contain a mixture of different aromatic polycarbonates, any preferential ranges are to be understood as an indication for the overall mixture of aromatic polycarbonates.

A portion of up to 80 mol %, preferably of 20 mol % to 50 mol %, of the carbonate groups in the polycarbonates used according to the invention may be replaced by aromatic dicarboxylic ester groups. Such polycarbonates which contain both acid radicals of carbonic acid and acid radicals of aromatic dicarboxylic acids incorporated in the molecular chain are referred to as aromatic polyester carbonates. In the context of the present invention they are encompassed by the umbrella term “thermoplastic aromatic polycarbonates”.

Particulars pertaining to the production of polycarbonates are disclosed in many patent documents spanning approximately the last 40 years. Reference may be made here for example to Schnell, “Chemistry and Physics of Polycarbonates”, Polymer Reviews, Volume 9, Interscience Publishers, New York, London, Sydney 1964, to D. Freitag, U. Grigo, P. R. Müller, H. Nouvertné, BAYER AG, “Polycarbonates” in Encyclopedia of Polymer Science and Engineering, Volume 11, Second Edition, 1988, pages 648-718, and lastly to U. Grigo, K. Kirchner and P. R. Müller “Polycarbonate” [Polycarbonates] in Becker/Braun, Kunststoff-Handbuch [Plastics Handbook], Volume 3/1, Polycarbonate, Polyacetale, Polyester, Celluloseester [Polycarbonates, polyacetals, polyesters, cellulose esters], Carl Hanser Verlag Munich, Vienna 1992, pages 117 to 299.

Aromatic polycarbonates are produced for example by reaction of dihydroxyaryl compounds with carbonyl halides, preferably phosgene, and/or with aromatic dicarbonyl dihalides, preferably benzenedicarbonyl dihalides, by the interfacial process, optionally with use of chain terminators and optionally with use of trifunctional or more than trifunctional branching agents. Production via a melt polymerization process by reaction of dihydroxyaryl compounds with, for example, diphenyl carbonate is likewise possible.

For production of polyester carbonates a portion of the carbonic acid derivatives is replaced by aromatic dicarboxylic acids or derivatives of dicarboxylic acids and, depending on the carbonate structural units to be replaced in the aromatic polycarbonates, specifically with aromatic dicarboxylic ester structural units.

Dihydroxyaryl compounds suitable for producing polycarbonates are those of formula (1)

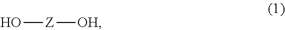

in which
Z is an aromatic radical which has 6 to 30 carbon atoms and may contain one or more aromatic rings, may be substituted and may contain aliphatic or cycloaliphatic radicals/alkylaryls or heteroatoms as bridging members.

It is preferable when Z in formula (1) is a radical of formula (2)

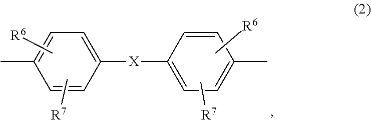

in which
R⁶ and R⁷ are independently H, C₁- to C₁₈-alkyl, C₁- to C₁₈-alkoxy, halogen such as Cl or Br or in each case optionally substituted aryl or aralkyl, preferably H or C₁- to C₁₂-alkyl, particularly preferably H or C₁- to C₈-alkyl and very particularly preferably H or methyl and
X is a single bond, —SO₂—, —CO—, —O—, —S—, C₁- to C₆-alkylene, C₂- to C₅-alkylidene or C₅- to C₆-cycloalkylidene which may be substituted with C₁- to C₆-alkyl, preferably methyl or ethyl, or else is C₆- to C₁₂-arylene which may optionally be fused to further aromatic rings containing heteroatoms.
X is preferably a single bond, C₁- to C₅-alkylene, C₂- to C₅-alkylidene, C₅- to C₆-cycloalkylidene, —O—, —SO—, —CO—, —S—, —SO₂—
or a radical of formula (3)

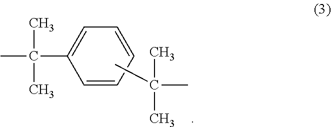

Examples of dihydroxyaryl compounds are: dihydroxybenzenes, dihydroxydiphenyls, bis(hydroxyphenyl)alkanes, bis(hydroxyphenyl)cycloalkanes, bis(hydroxyphenyl) aryls, bis(hydroxyphenyl) ethers, bis(hydroxyphenyl) ketones, bis(hydroxyphenyl) sulfides, bis(hydroxyphenyl) sulfones, bis(hydroxyphenyl) sulfoxides, 1,1′-bis(hydroxyphenyl)-diisopropylbenzenes and the ring-alkylated and ring-halogenated compounds thereof.

Dihydroxyaryl compounds suitable for producing polycarbonates include for example hydroquinone, resorcinol, dihydroxydiphenyls, bis(hydroxyphenyl)alkanes, bis(hydroxyphenyl)cycloalkanes, bis(hydroxyphenyl) sulfides, bis(hydroxyphenyl) ethers, bis(hydroxyphenyl) ketones, bis(hydroxyphenyl) sulfones, bis(hydroxyphenyl) sulfoxides, α,α′-bis(hydroxyphenyl)-diisopropylbenzenes, phthalimidines derived from derivatives of isatin or phenolphthalein, and the ring-alkylated, ring-arylated and ring-halogenated compounds thereof.

Preferred dihydroxyaryl compounds are 4,4′-dihydroxydiphenyl, 2,2-bis(4-hydroxyphenyl) propane (bisphenol A), 2,4-bis(4-hydroxyphenyl)-2-methylbutane, 1,1-bis(4-hydroxyphenyl)-p-diisopropylbenzene, 2,2-bis(3-methyl-4-hydroxyphenyl)propane, dimethylbisphenol A, bis(3,5-dimethyl-4-hydroxyphenyl) methane, 2,2-bis(3,5-dimethyl-4-hydroxyphenyl)propane, bis(3,5-dimethyl-4-hydroxyphenyl) sulfone, 2,4-bis(3,5-dimethyl-4-hydroxyphenyl)-2-methylbutane, 1,1-bis(3,5-dimethyl-4-hydroxyphenyl)-p-diisopropylbenzene and 1,1-bis(4-hydroxyphenyl)-3,3,5-trimethylcyclohexane and also the dihydroxyaryl compounds (I) to (III)

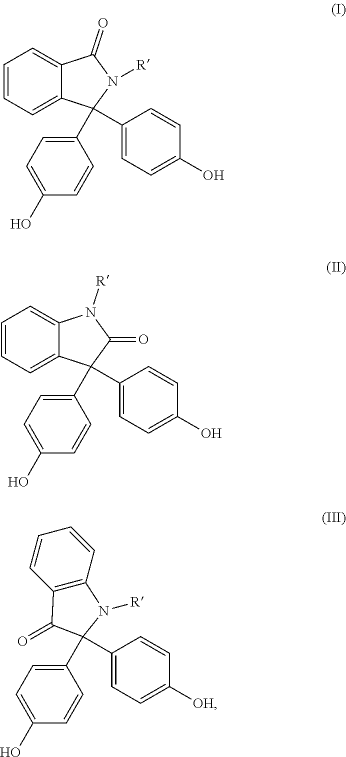

in which each R′is a C₁- to C₄-alkyl radical, aralkyl radical or aryl radical, preferably a methyl radical or phenyl radical, very particularly preferably a methyl radical.

Particularly preferred dihydroxyaryl compounds are 2,2-bis(4-hydroxyphenyl)propane (bisphenol A), 2,2-bis(3,5-dimethyl-4-hydroxyphenyl)propane, 1,1-bis(4-hydroxyphenyl)cyclohexane, 1,1-bis(4-hydroxyphenyl)-3,3,5-trimethylcyclohexane, 4,4′-dihydroxydiphenyl and dimethyl-bisphenol A, and also the bisphenols of formulae (I), (II) and (III).

These and other suitable dihydroxyaryl compounds are described by way of example in U.S. Pat. Nos. 3,028,635 A, 2,999,835 A, 3,148,172 A, 2,991,273 A, 3,271,367 A, 4,982,014 A and 2,999,846 A, in DE 1 570 703 A, DE 2063 050 A, DE 2 036 052 A, DE 2 211 956 A and DE 3 832 396 A, in FR 1 561 518 A, in the monograph “H. Schnell, Chemistry and Physics of Polycarbonates, Interscience Publishers, New York 1964”, and in JP 62039/1986 A, JP 62040/1986 A and JP 105550/1986 A.

In the case of the homopolycarbonates, only one dihydroxyaryl compound is used; in the case of the copolycarbonates, two or more dihydroxyaryl compounds are used.

Examples of suitable carbonic acid derivatives include phosgene or diphenyl carbonate.

Suitable chain terminators that may be employed in the production of the polycarbonates include monophenols. Examples of suitable monophenols include phenol itself, alkylphenols such as cresols, p-tert-butylphenol, cumylphenol, and mixtures thereof.

Preferred chain terminators are the phenols which are mono- or polysubstituted with linear or branched, preferably unsubstituted, C₁- to C₃₀-alkyl radicals, or with tert-butyl. Particularly preferred chain terminators are phenol, cumylphenol and/or p-tert-butylphenol.

The amount of chain terminator to be used is preferably 0.1 to 5 mol %, based on moles of dihydroxyaryl compounds used in each case. The chain terminators may be added before, during or after the reaction with a carbonic acid derivative.

Suitable branching agents are the trifunctional or more than trifunctional compounds known in polycarbonate chemistry, in particular those having three or more than three phenolic OH groups.

Examples of suitable branching agents include 1,3,5-tri(4-hydroxyphenyl)benzene, 1,1,1-tri(4-hydroxyphenyl)ethane, tri(4-hydroxyphenyl)phenylmethane, 2,4-bis(4-hydroxyphenyl-isopropyl)phenol, 2,6-bis(2-hydroxy-5′-methylbenzyl)-4-methylphenol, 2-(4-hydroxyphenyl)-2-(2,4-dihydroxyphenyl)propane, tetra(4-hydroxyphenyl)methane, tetra(4-(4-hydroxyphenyl-isopropyl)phenoxy)methane and 1,4-bis((4′,4″-dihydroxytriphenyl) methyl)benzene and 3,3-bis(3-methyl-4-hydroxyphenyl)-2-oxo-2,3-dihydroindole.

The amount of any branching agents to be used is preferably 0.05 mol % to 2.00 mol %, based on moles of dihydroxyaryl compounds used in each case.

The branching agents can either form an initial charge with the dihydroxyaryl compounds and the chain terminators in the aqueous alkaline phase or can be added, dissolved in an organic solvent, before the phosgenation. In the case of the transesterification method, the branching agents are used together with the dihydroxyaryl compounds.

Particularly preferred polycarbonates are the homopolycarbonate based on bisphenol A, the homopolycarbonate based on 1,1-bis(4-hydroxyphenyl)-3,3,5-trimethylcyclohexane and the copolycarbonates based on the two monomers bisphenol A and 1,1-bis(4-hydroxyphenyl)-3,3,5-trimethylcyclohexane or the two monomers bisphenol A and 4,4′-dihydroxydiphenyl, and homo- or copolycarbonates derived from the dihydroxyaryl compounds of formulae (I), (II) and/or (III)

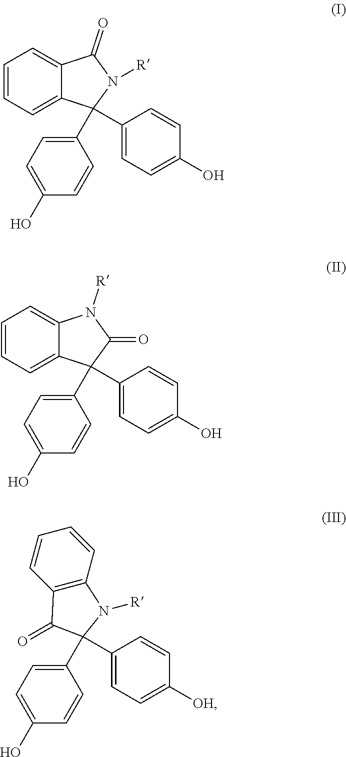

in which each R′ is C₁- to C₄-alkyl, aralkyl or aryl, preferably methyl or phenyl, very particularly preferably methyl,
   especially with bisphenol A. Very particularly preferably, the aromatic polycarbonate comprises a bisphenol A-based homopolycarbonate. Exceptionally preferably, the aromatic polycarbonate is bisphenol A-based homopolycarbonate.

The total proportion of the monomer units based on formulae (I), (II), (III), 4,4′-dihydroxydiphenyl and/or bisphenol TMC in the copolycarbonate is preferably 0.1-88 mol %, particularly preferably 1-86 mol %, very particularly preferably 5-84 mol % and in particular 10-82 mol % (based on the sum total of the moles of dihydroxyaryl compounds used).

The relative solution viscosity of the copolycarbonates, determined according to ISO 1628-4:1999, is preferably in the range of 1.15-1.35.

The dihydroxyaryl compounds used, like all the other chemicals and auxiliaries added to the synthesis, may be contaminated with the impurities originating from their own synthesis, handling and storage. However, it is desirable to employ raw materials of the highest possible purity.

Also preferred are copolycarbonates produced using diphenols of general formula (4a):

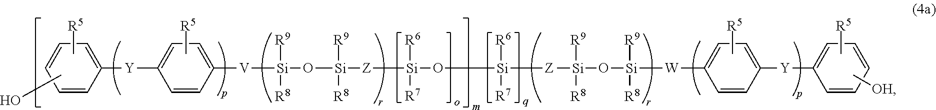

where
R⁵ is hydrogen or C₁- to C₄-alkyl, C₁- to C₃-alkoxy, preferably hydrogen; methoxy or methyl,
R^{6,} R^{7,} R⁸ and R⁹ are each independently C₁- to C₄-alkyl or C₆- to C₁₂-aryl, preferably methyl or phenyl,
Y is a single bond, SO₂—, —S—, —CO—, —O—, C₁- to C₆-alkylene, C₂- to C₅-alkylidene, C₆- to C₁₂-arylene which may optionally be fused to further aromatic rings containing heteroatoms or is a C₅- to C₆-cycloalkylidene radical which may be mono- or polysubstituted by C₁- to C₄-alkyl, preferably is a single bond, —O—, isopropylidene or a C₅- to C₆-cycloalkylidene radical which may be mono- or polysubstituted by C₁- to C₄-alkyl,
V is oxygen, C₂- to C₆-alkylene or C₃- to C₆-alkylidene, preferably oxygen or C₃-alkylene,
p, q and r are each independently 0 or 1,
when q=0, W is a single bond, when q=1 and r=0, W is oxygen, C₂- to C₆-alkylene or C₃- to C₆-alkylidene, preferably oxygen or C₃-alkylene,
when q=1 and r=1, W and V are each independently C₂- to C₆-alkylene or C₃- to C₆-alkylidene, preferably C₃-alkylene,

Z is a C₁- to C₆-alkylene, preferably C₂-alkylene,
o is an average number of repeat units from 10 to 500, preferably 10 to 100, and
m is an average number of repeat units from 1 to 10, preferably 1 to 6, further preferably 1.5 to 5. It is likewise possible to use diphenols in which two or more siloxane blocks of general formula (4a) are joined to one another via terephthalic acid and/or isophthalic acid to form ester groups.

Especially preferred are (poly) siloxanes of formulae (5) and (6)

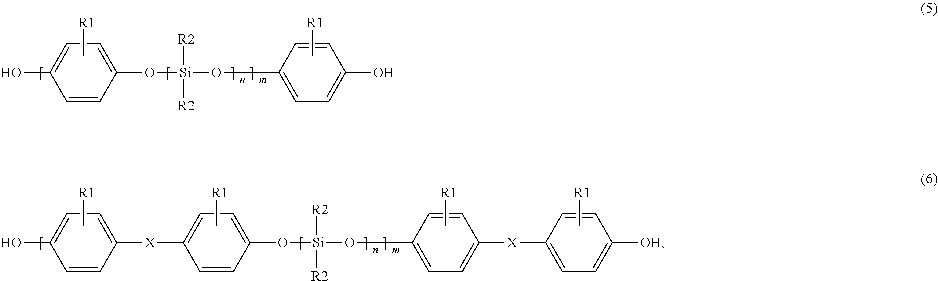

where R1 is hydrogen, C₁ to C₄ alkyl, preferably hydrogen or methyl, and especially preferably hydrogen,
R2 is independently aryl or alkyl, preferably methyl,
X is a single bond, —SO₂—, —CO—, —O—, —S—, C₁ to C₆ alkylene, C₂ to C₅ alkylidene or C₆ to C₁₂ arylene, which may optionally be fused with further aromatic rings containing heteroatoms,
X is preferably a single bond, C₁ to C₅ alkylene, C₂ to C₅ alkylidene, C₅ to C₁₂ cycloalkylidene, —O—, —SO—, —CO—, —S—, —SO₂—, particularly preferably X is a single bond, isopropylidene, C₅ to C₁₂ cycloalkylidene or oxygen, and very particularly preferably is isopropylidene,
n is an average number from 10 to 400, preferably 10 to 100, especially preferably 15 to 50, and
m is an average number from 1 to 10, preferably from 1 to 6 and especially preferably from 1.5 to 5.

The siloxane block may likewise preferably be derived from the following structure

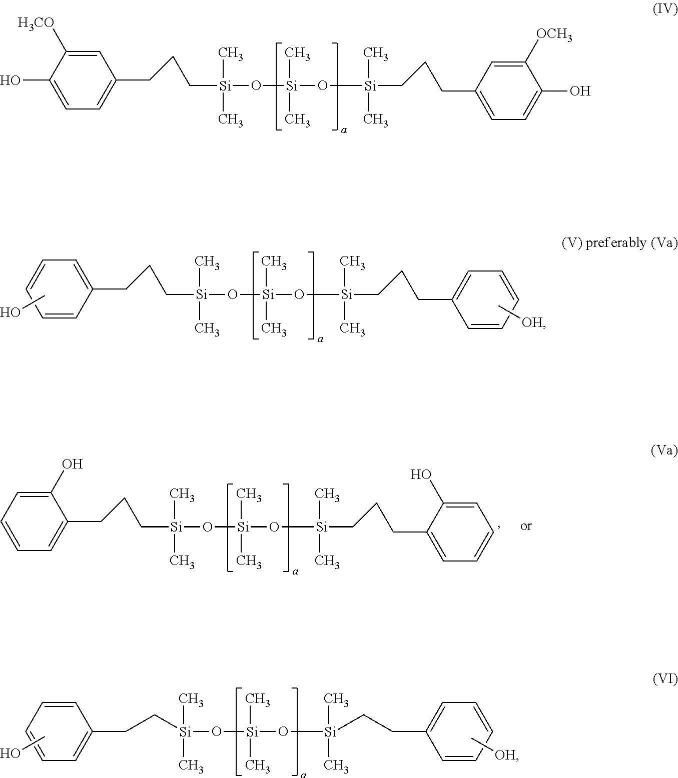

where a in formulae (IV), (V) and (VI) is an average number from 10 to 400, preferably 10 to 100 and particularly preferably 15 to 50.

It is likewise preferable when at least two identical or different siloxane blocks of general formulae (IV), (V) or (VI) are joined to one another via terephthalic acid and/or isophthalic acid to form ester groups.

It is likewise preferable when, in formula (4a), p=0, V is C₃-alkylene, r=1, Z is C₂-alkylene, R⁸ and R° are methyl, q=1, W is C₃-alkylene, m=1, R⁵ is hydrogen or C₁- to C₄-alkyl, preferably hydrogen or methyl, R⁶ and R⁷ are each independently C₁- to C₄-alkyl, preferably methyl, and o is 10 to 500.

Copolycarbonates having monomer units of formula (4a) and in particular also the production thereof are described in WO 2015/052106 A2.

Copolycarbonates having monomer units of formula (IV) and in particular also the production thereof are described in WO 2015/052106 A2.

Examples of aromatic dicarboxylic acids that are suitable for the production of the polyestercarbonates include orthophthalic acid, terephthalic acid, isophthalic acid, tert-butylisophthalic acid, 3,3′-diphenyldicarboxylic acid, 4,4′-diphenyldicarboxylic acid, 4,4-benzophenonedicarboxylic acid, 3,4′-benzophenonedicarboxylic acid, 4,4′-diphenyl ether dicarboxylic acid, 4,4′-diphenylsulfonedicarboxylic acid, 2,2-bis(4-carboxyphenyl)propane, trimethyl-3-phenylindane-4,5′-dicarboxylic acid.

Among the aromatic dicarboxylic acids, particular preference is given to using terephthalic acid and/or isophthalic acid.

Derivatives of dicarboxylic acids are dicarbonyl dihalides and dialkyl dicarboxylates, especially dicarbonyl dichlorides and dimethyl dicarboxylates.

Replacement of the carbonate groups by the aromatic dicarboxylic ester groups is substantially stoichiometric, and also quantitative, and the molar ratio of the reactants is therefore also maintained in the final polyester carbonate. The aromatic dicarboxylic ester groups may be incorporated either randomly or in blocks.

The compositions according to the invention contain at least 75% by weight, preferably at least 85% by weight, more preferably at least 90% by weight, particularly preferably at least 95% by weight, exceptionally preferably at least 97.5% by weight, of aromatic polycarbonate, and are thus based on aromatic polycarbonate.

### Component B

Component B is a functionalized siloxane polymer composed only of siloxane units as monomer units, whose group of monomer units thus consists of siloxane units. Component B may be a single siloxane polymer or a mixture of two or more siloxane polymers. It is preferable when the siloxane polymer—if it is a mixture of different siloxane polymers—has a weight-average molecular weight M_{w} of >500 000 g/mol, more preferably of >750 000 g/mol, particularly preferably of >1 000 000 g/mol, determined by GPC in tetrahydrofuran (THF) calibrated against polystyrene standards, and is thus a UHMW (ultra high molecular weight) siloxane polymer.

In the present case “composed only of . . . ” is to be understood as meaning that these polymers are based on no further monomer units. They are linear or branched polymers or copolymers, wherein preferably the organic groups of the siloxane units are independently selected from methyl or phenyl groups. Suitable siloxane polymers of component B include polydimethylsiloxane homopolymers and copolymers composed of dimethylsiloxane and methylphenylsiloxane units, copolymers composed of dimethylsiloxane and diphenylsiloxane units, copolymers composed of diphenylsiloxane units and methylphenylsiloxane units and homopolymers of methylpheny lsiloxane units. Mixtures of two or more of these polymers or copolymers may also be used for blends of the above-described higher and/or lower molecular weight siloxane polymer. It is particularly preferable when component B is at least one polymer selected from the group consisting of polydimethylsiloxane homopolymers and copolymers composed of dimethylsiloxane and methylphenylsiloxane units, copolymers composed of dimethylsiloxane and diphenylsiloxane units, copolymers composed of diphenylsiloxane units and methylphenylsiloxane units, homopolymers of methylphenylsiloxane units or mixtures of the aforementioned polymers.

The siloxane polymer of component B is a functionalized siloxane polymer. “Functionalized” in this context is to be understood as meaning that the siloxane polymer in its entirety comprises one or more reactive groups. According to the invention, “reactive groups” is to be understood here as meaning groups that fundamentally determine the substance properties and the reaction behavior of the compound. Such reactive groups include for example groups containing hydroxyl, methyl, fluoro, carboxyl or nitrogen-containing groups such as amine or alkenyl groups. It is particularly preferable when the siloxane polymer comprises hydroxyl groups. “In its entirety” is to be understood as meaning that not every siloxane polymer must comprise groups corresponding to the mixture but that siloxane polymer comprising reactive groups must be present. It is preferable when the entire siloxane polymer according to component B comprises one or more representatives of reactive groups, in particular at least hydroxyl groups.

The reactive groups are arranged at the end of the molecule and/or along the polymer chain, preferably at the chain ends. It is very particularly preferable when the reactive groups arranged at the chain ends are hydroxyl groups, exceptionally preferably in the form of diorganohydroxysiloxy groups, for example dimethylhydroxysiloxy, diphenylhydroxysiloxy and/or methylphenylhydroxysiloxy groups.

If the reactive groups are arranged only along the polymer chain, the terminal groups of the siloxane polymer are not reactive and typically a di- or triorganosiloxy species, for example dimethylvinylsiloxy or trimethylsiloxy groups.

It is particularly preferable when component B comprises hydroxyl-terminated poly dimethylsiloxane and component B is very particularly preferably hydroxyl-terminated polydimethylsiloxane.

The siloxane polymer of component B is exceptionally preferably functionalized linear polydimethylsiloxane containing up to 50 mol % methyl radicals, preferably polydimethylsiloxane homopolymer with dimethylhydroxysiloxy end groups.

### Component C

The thermoplastic compositions employed according to the invention may also contain one or more additives, referred to here as component C. The term “may” is to be understood as meaning that a further additive need not be present and the amount of component C may thus also be 0% by weight. The amount of further additives is preferably up to 15% by weight, more preferably up to 10% by weight, particularly preferably up to 5% by weight, very particularly preferably up to 1.0% by weight.

Such further additives, such as are typically added to polycarbonates, include in particular heat stabilizers, radiation stabilizers, flame retardants, antioxidants, demolding agents, anti-drip agents, for instance polytetrafluoroethylene (Teflon) or SAN-encapsulated PTFE (e.g. Blendex 449), UV absorbers, IR absorbers, impact modifiers, optical brighteners, fillers, for example talc, silicates or quartz, light scattering agents, hydrolysis stabilizers, transesterification stabilizers, compatibilizers, organic dyes, organic pigments, inorganic pigments and/or additives for laser marking, especially in the amounts customary for polycarbonate-based compositions. Such additives are described for example in EP-A 0 839 623, WO-A 96/15102, EP-A 0 500 496 or in “Plastics Additives Handbook”, Hans Zweifel, 5th Edition 2000, Hanser Verlag, Munich. These additives may be added individually or else in admixture.

It will be appreciated that it is permissible to add only additives of such a nature and in such amounts that they do not have a significant adverse impact on the effect according to the invention of improving intralipid resistance.

It is preferable when the thermoplastic compositions contain one or more demolding agents, one or more heat stabilizers and/or antioxidants and optionally colorants as further additives.

Suitable demolding agents especially include those based on a fatty acid ester, yet more preferably on a stearic ester, especially preferably on pentaerythritol. Particular preference is given to using pentaerythritol tetrastearate (PETS) and/or glycerol monostearate (GMS). When one or more demolding agents are employed the amount is preferably up to 1.0% by weight (inclusive), more preferably 0.01% to 0.7% by weight, particularly preferably 0.02% to 0.60% by weight, in each case based on the total composition.

If heat stabilizer is employed the amount is preferably up to 0.20% by weight, more preferably 0.01% to 0.10% by weight, yet more preferably 0.01% to 0.05% by weight, particularly preferably 0.015% to 0.040% by weight, based on the total composition.

Suitable heat stabilizers are in particular phosphorus-based stabilizers selected from the group of the phosphates, phosphites, phosphonites, phosphines and mixtures thereof. Examples include triphenyl phosphite, diphenyl alkyl phosphite, phenyl dialkyl phosphite, tris(nonylphenyl) phosphite, trilauryl phosphite, trioctadecyl phosphite, distearyl pentaerythritol diphosphite, tris(2,4-di-tert-butylphenyl) phosphite (Irgafos® 168), diisodecyl pentaerythritol diphosphite, bis(2,4-di-tert-butylphenyl) pentaerythritol diphosphite, bis(2,4-dicumylphenyl) pentaerythritol diphosphite (Doverphos® S-9228), bis(2,6-di-tert-butyl-4-methylphenyl) pentaerythritol diphosphite, diisodecyloxy pentaerythritol diphosphite, bis(2,4-di-tert-butyl-6-methylphenyl) pentaerythritol diphosphite, bis(2,4,6-tris(tert-butylphenyl) pentaerythritol diphosphite, tristearyl sorbitol triphosphite, tetrakis (2,4-di-tert-butylphenyl)-4,4′-biphenylenediphosphonite, 6-isooctyloxy-2,4,8,10-tetra-tert-butyl-12H-dibenzo[d,g]-1,3,2-dioxaphosphocine, bis(2,4-di-tert-butyl-6-methylphenyl) methyl phosphite, bis(2,4-di-tert-butyl-6-methylphenyl) ethyl phosphite, 6-fluoro-2,4,8,10-tetra-tert-butyl-12-methyldibenzo[d,g]-1,3,2-dioxaphosphocine, 2,2′,2″-nitrilo[triethyltris(3,3′,5,5′-tetra-tert-butyl-1,1′-biphenyl-2,2′-diyl) phosphite], 2-ethylhexyl (3,3′,5,5′-tetra-tert-butyl-1,l′-biphenyl-2,2′-diyl) phosphite, 5-butyl-5-ethyl-2-(2,4,6-tri-tert-butylphenoxy)-1,3,2-dioxaphosphirane, bis(2,6-di-tert-butyl-4-methylphenyl) pentaerythritol diphosphite, triphenylphosphine (TPP), trialkylphenylphosphine, bisdiphenylphosphinoethane or a trinaphthylphosphine. They are used alone or in a mixture, for example Irganox® B900 (mixture of Irgafos® 168 and Irganox® 1076 in a 4:1 ratio) or Doverphos® S-9228 with Irganox® B900 or Irganox® 1076. Especially preferably, triphenylphosphine (TPP), Irgafos® 168 or tris(nonylphenyl) phosphite, or mixtures thereof, are used.

It is also possible to use phenolic antioxidants such as alkylated monophenols, alkylated thioalkylphenols, hydroquinones and alkylated hydroquinones. It is particularly preferable to employ Irganox® 1010 (pentaerythritol-3-(4-hydroxy-3,5-di-tert-butylphenyl) propionate; CAS: 6683-19-8) and Irganox 1076® (octadecyl-3-(3,5-di-tert-butyl-4-hydroxyphenyl)-propionate), preferably in amounts of 0.05%-0.5% by weight.

It is also possible to add sulfonic esters or alkyl phosphates, for example mono-, di- and/or trihexyl phosphate, triisooctyl phosphate and/or trinonyl phosphate, as transesterification inhibitors. If transesterification stabilizer is present the alkyl phosphate used is preferably triisooctyl phosphate (tris-2-ethylhexyl phosphate). It is also possible to use mixtures of various mono-, di- and trialkyl phosphates. Triisooctyl phosphate is preferably used in amounts from 0.003% by weight to 0.05% by weight, further preferably 0.005% by weight to 0.04% by weight and particularly preferably from 0.01% by weight to 0.03% by weight, based on the overall composition.

The compositions used for the medical devices according to the invention are typically produced as follows:

For incorporation of additives the aromatic polycarbonate of component A, on which the thermoplastic composition is based, is employed optionally as a thermoplastic mixture, preferably in the form of powders, pellets or of mixtures of powders and pellets.

Other than the additives the compositions employed according to the invention may optionally also comprise one or more blend partners, optionally in addition to the additives. Suitable blend partners include ABS, polyesters such as PBT or PET, PMMA or mixtures thereof.

Component B as well as the further constituents, i.e. especially the further additives, are converted into corresponding compositions by commonplace methods of incorporation by combining, mixing and homogenizing the individual constituents, wherein especially the homogenization is preferably carried out in the melt by application of shear forces. Combination and mixing is optionally effected prior to melt homogenization using powder premixes.

It is also possible to use premixes of pellets or pellets and powders with the individual components.

It is also possible to use premixes produced from solutions of the mixture components in suitable solvents where homogenization is optionally effected in solution and the solvent is then removed.

The components of the compositions may especially be introduced into the thermoplastic polymer, in particular into the aromatic polycarbonate, optionally into the aromatic polycarbonate with blend partners, by known methods or as a masterbatch. It is preferable when at least component B is introduced into the thermoplastic composition as a masterbatch in aromatic polycarbonate, more preferably in bisphenol A-based homopolycarbonate.

The use of masterbatches, based on aromatic polycarbonate in the present case, is preferred for introducing the respective components, individually or in admixture. This especially also applies to component B, the siloxane polymer. When introduced by means of a masterbatch said component preferably contains 0.5% to 60% by weight, more preferably 20% to 55% by weight, particularly preferably 45% to 52% by weight, of siloxane polymer based on the total weight of the masterbatch. Introduction as a masterbatch typically makes it possible to achieve a homogeneous distribution of the additive.

The composition employed according to the invention may be combined, mixed, homogenized and subsequently extruded in customary apparatuses such as screw extruders (for example ZSK twin-screw extruders), kneaders or Brabender or Banbury mills. The extrudate may be cooled and comminuted after extrusion. It is also possible to premix individual components and then to add the remaining starting materials individually and/or likewise mixed.

The combining and mixing of a premix in the melt may also be effected in the plasticizing unit of an injection molding machine. This comprises directly converting the melt into a shaped article in the subsequent step.

The compositions may be processed into any desired shaped articles in customary fashion in customary machines, for example in extruders or injection molding machines.

Preference according to the invention is given to a
medical device or part of a medical device,
wherein the medical device or part of a medical device comprises an element consisting of a thermoplastic composition containing the following components:
A) at least 90% by weight of aromatic polycarbonate,
B) 0.2% to 1.5% by weight of functionalized siloxane polymer composed only of siloxane units as monomer units, wherein the functionalized siloxane polymer has a weight-average molecular weight M_{w}, determined by GPC in tetrahydrofuran (THF) calibrated against polystyrene standards, of >500 000 g/mol and comprises dimethylsiloxane units and hydroxyl groups,
   wherein the reported amounts are based on the total weight of the thermoplastic composition and
   wherein the element is intended to be in contact with intralipid during the intended use of the medical device.

Further preference according to the invention is given to a
medical device or part of a medical device,
wherein the medical device or part of a medical device comprises an element consisting of a thermoplastic composition consisting of the following components:
A) at least 90% by weight of aromatic polycarbonate,
B) 0.2% to 1.5% by weight of functionalized siloxane polymer composed only of siloxane units as monomer units,
   wherein the functionalized siloxane polymer preferably has a weight-average molecular weight M_{w}, determined by GPC in tetrahydrofuran (THF) calibrated against polystyrene standards, of >500 000 g/mol and comprises dimethylsiloxane units and hydroxyl groups,
C) optionally one or more further additives selected from the group consisting of heat stabilizers, flame retardants, antioxidants, demolding agents, anti-drip agents, UV absorbers, IR absorbers, impact modifiers, optical brighteners, fillers, light-scattering agents, hydrolysis stabilizers, transesterification stabilizers, compatibilizers, organic dyes, organic pigments, inorganic pigments and/or additives for laser marking,
wherein the reported amounts are based on the total weight of the thermoplastic composition and wherein the element is intended to be in contact with intralipid during the intended use of the medical device.

Particular preference according to the invention is given to a
medical device or part of a medical device,
wherein the medical device or part of a medical device comprises an element consisting of a thermoplastic composition consisting of the following components:
A) at least 90% by weight of aromatic polycarbonate,
B) 0.2% to 1.5% by weight of functionalized siloxane polymer composed only of siloxane units as monomer units,
   wherein the functionalized siloxane polymer preferably has a weight-average molecular weight M_{w}, determined by GPC in tetrahydrofuran (THF) calibrated against polystyrene standards, of >500 000 g/mol and comprises dimethylsiloxane units and hydroxyl groups,
C) optionally one or more further additives selected from the group consisting of demolding agents, heat stabilizers, antioxidants, colorants, pigments,
wherein the reported amounts are based on the total weight of the thermoplastic composition and wherein the element is intended to be in contact with intralipid during the intended use of the medical device.

Very particular preference according to the invention is given to a
medical device or part of a medical device,
wherein the medical device or part of a medical device comprises an element consisting of a thermoplastic composition consisting of the following components:
A) at least 90% by weight of aromatic polycarbonate, wherein the aromatic polycarbonate preferably contains bisphenol A-homopolycarbonate, particularly preferably is bisphenol A-homopolycarbonate, and
B) 0.2% to 1.5% by weight, in particular 0.5% to 1% by weight, of functionalized siloxane polymer composed only of siloxane units as monomer units,
   wherein the functionalized siloxane polymer preferably has a weight-average molecular weight M_{w}, determined by GPC in tetrahydrofuran (THF) calibrated against polystyrene standards, of >500 000 g/mol and comprises dimethylsiloxane units and hydroxyl groups,
C) optionally one or more further additives selected from the group consisting of demolding agents, heat stabilizers, antioxidants, colorants, pigments,
wherein the reported amounts are based on the total weight of the thermoplastic composition and wherein the element is intended to be in contact with intralipid during the intended use of the medical device.

Greatest preference is given to a
medical device or part of a medical device,
wherein the medical device or part of a medical device comprises an element consisting of a thermoplastic composition consisting of the following components:
A) at least 90% by weight of aromatic polycarbonate, wherein the aromatic polycarbonate is bisphenol A-homopolycarbonate and has a melt volume flow rate MVR of 16 to 20 cm³/(10 min), determined according to ISO 1133:2012-03, at a test temperature of 300° C. and a load of 1.2 kg, and
B) 0.2% to 1.5% by weight, in particular 0.5% to 1% by weight, of functionalized siloxane polymer composed only of siloxane units as monomer units,
   wherein the functionalized siloxane polymer has a weight-average molecular weight M_{w}, determined by GPC in tetrahydrofuran (THF) calibrated against polystyrene standards, of >500 000 g/mol and comprises dimethylsiloxane units and hydroxyl groups,
C) optionally one or more further additives selected from the group consisting of demolding agents, heat stabilizers, antioxidants, colorants, pigments,
wherein the reported amounts are based on the total weight of the thermoplastic composition and wherein the element is intended to be in contact with intralipid during the intended use of the medical device.

In the preferred, more preferred etc. embodiments described above the functionalized siloxane polymer is particularly preferably a dimethyl siloxane homopolymer functionalized at least with hydroxyl groups, very particularly preferably functionalized exclusively with hydroxyl groups.

The embodiments described above as preferred, more preferred etc. for the medical device/part of a medical device according to the invention also apply—if applicable—to the use according to the invention of component B, i.e. the use for improving the intralipid resistance of compositions based on aromatic polycarbonate which preferably contain at least 75% by weight of aromatic polycarbonate, more preferably at least 90% by weight of aromatic polycarbonate.

The examples which follow are intended to illustrate the invention, without limiting said invention.

### EXAMPLES

### 1. Components

Component A-1: Linear homopolycarbonate based on bisphenol A having a melt volume flow rate (MVR) of 19 cm³/(10 min) (according to ISO 1133:2012-03 at a test temperature of 300° C. and a test load of 1.2 kg).
Component A-2: Linear homopolycarbonate based on bisphenol A having a melt volume flow rate (MVR) of 6 cm³/(10 min) (according to ISO 1133:2012-03 at a test temperature of 300° C. and a test load of 1.2 kg).
Component A-3: Linear homopolycarbonate based on bisphenol A having a melt volume flow rate (MVR) of 5 cm³/(10 min) (according to ISO 1133:2012-03 at a test temperature of 300° C. and a test load of 1.2 kg).
Component B*-1: Pelletized composition containing 50% by weight of hydroxyl end group-functionalized UHMW siloxane polymer (polydimethylsiloxane) dispersed in aromatic polycarbonate. Component B, functionalized siloxane polymer, is thus present in examples 2 and 3 in an amount of 0.5% by weight and 1% by weight respectively based on the total composition.
Component C-1: Demolding agent pentaerythritol tetrastearate from Emery Oleochemicals.
Component C-2: Additive package of multranol 3600 DHP (alpha,omega-bis(tetrahydro-2H-pyran-2-yl)-poly[oxy(methyl-1,2-ethanediyl)], polyether polyol. Mₙ=2000 g/mol) and two anthraquinone dyes.
Employed intralipid: SmofKabiven electrolyte-free emulsion from Fresenius Kabi AG. Active ingredients: 1000 ml contains: Refined soybean oil (Ph.Eur.) 11.4 g, medium chain triglycerides 11.4 g, refined olive oil 9.5 g, omega-3 acid-rich fish oil 5.7 g, glucose (as glucose monohydrate (Ph.Eur.)) 127 g, alanine 7.1 g, arginine 6.1 g, glycine 5.6 g, histidine 1.5 g, isoleucine 2.5 g, leucine 3.8 g, lysine acetate 3.4 g, methionine 2.2 g, phenylalanine 2.6 g, proline 5.7 g, serine 3.3 g, taurine 0.5 g, threonine 2.2 g, tryptophan 1.0 g, tyrosine 0.20 g, valine 3.1 g, corresponding to amino acids 51 g, nitrogen 8 g, carbohydrates (glucose anhydrous) 127 g, lipids 38 g, acetate (content from amino acid solution) 74.5 mmol, phosphate (content from lipid emulsion) 2.8 mmol. Total energy about 1100 kcal (4.6 MJ), non-protein energy about 900 kcal (3.8 MJ). Osmolality about 1600 mosm/kg water, osmolarity about 1300 mosm/l, pH (after mixing) about 5.6. Other constituents: glycerol, egg lecithin, alpha-tocopherol (Ph.Eur.), sodium hydroxide, sodium oleate, acetic acid 99%, hydrochloric acid 10%, water for injection. For intravenous infusion for parenteral nutrition.

### Procedure

The polycarbonate compositions described in the following examples were produced on a Berstorff ZE 25 extruder at a throughput of 10 kg/h by compounding. The melt temperature was 275° C.

Environmental stress cracking (ESC) is used as a measure for chemicals resistance. ESC was determined by the bending strip method at room temperature. A test specimen measuring 80 mm×10 mm×4 mm injection molded at a melt temperature of 280° C. and a mold temperature of 80° C. was subjected to an outer fiber stress (OFS) of 1.4% with a bending jig. Immediately after clamping of the test specimen the test specimen was contacted with the test medium (SmofKabiven electrolyte-free emulsion). To this end the test medium was applied dropwise to a fabric-like paper which was then placed on the specimen (centrally in the tensile zone of the test specimen). The clamped specimens were stored with test medium for one day/four days. The test specimens were then removed from the bending jig and subjected to a visual inspection to determine the surface condition of the test specimens according to the criteria described in table 1:

TABLE 1
Criteria for assessing chemicals resistance

| | | Abbreviated |
|---|---|---|
| Property | Failure criterion | description |
| Surface condition (assessed | Cracks or hairline cracks at | A1 |
| by visual inspection) | the edges of the stretched | |
| | surface | |
| | Cracks or hairline cracks on | A2 |
| | the stretched surface | |
| | No change | kV |

TABLE 2
Tests performed and results

| | | 1 | | |
|---|---|---|---|---|
| | | (comp.) | 2 | 3 |
| Components | | | | |
| A-1 | [% by wt.] | — | 98.6 | 97.6 |
| A-2 | [% by wt.] | 59.35 | — | — |
| A-3 | [% by wt.] | 40.0 | — | — |
| B*-1 | [% by wt.] | — | 1.0 | 2.0 |
| C-1 | [% by wt.] | — | 0.4 | 0.4 |
| C-2 | [% by wt.] | 0.65 | — | — |
| Chemicals resistance (ESC) at | | | | |
| 1.4% OFS | | | | |
| Surface condition after 1 day | | kV | kV | kV |
| of storage | | | | |
| Surface condition after 4 days | | A1 | kV | kV |
| of storage | | | | |

It is apparent from table 1 that only the inventive compositions of examples 2 and 3 solve the underlying problem but not the composition of comparative example 1 which is the conventional polycarbonate having an altogether high molecular weight and high chemicals resistance, i.e. only the inventive compositions of examples 2 and 3 exhibit a good intralipid resistance even upon prolonged exposure and thus show no change in their surface.

1. A medical device or part of a medical device, wherein the medical device or part of a medical device comprises an element consisting of a thermoplastic composition containing the following components:
A) at least 75% by weight of aromatic polycarbonate and
B) 0.2 to 1.5% by weight of functionalized siloxane polymer composed only of siloxane units as monomer units, wherein the reported amounts are based on the total weight of the thermoplastic composition and
wherein the element is intended to be in contact with intralipid during the intended use of the medical device.

2. The medical device or part of a medical device as claimed in claim 1, wherein the medical device is intended for use in parenteral nutrition.

3. The medical device or part of a medical device as claimed in claim 1, wherein the medical device or part of a medical device is a tube connector, a three-way stopcock, a manifold, a drip chamber, a Luer connector, an IV catheter.

4. The medical device or part of a medical device as claimed in claim 1, wherein the aromatic polycarbonate is bisphenol A-based homopolycarbonate.

5. The medical device or part of a medical device as claimed in claim 1, wherein the aromatic polycarbonate has a melt volume flow rate MVR of 15 to 35 cm³/(10 min), determined according to ISO 1133:2012-03, at a test temperature of 300° C. and a load of 1.2 kg.

6. The medical device or part of a medical device as claimed in claim 1, wherein the thermoplastic composition consists of the following components:
A) at least 90% by weight of aromatic polycarbonate,
B) 0.2% to 1.5% by weight of functionalized siloxane polymer composed only of siloxane units as monomer units,
and
C) optionally one or more further additives selected from the group consisting of heat stabilizers, flame retardants, antioxidants, radiation stabilizers, demolding agents, anti-drip agents, UV absorbers, IR absorbers, impact modifiers, optical brighteners, fillers, light-scattering agents, hydrolysis stabilizers, transesterification stabilizers, compatibilizers, organic dyes, organic pigments, inorganic pigments and/or additives for laser marking,
wherein the reported amounts are based on the total weight of the thermoplastic composition.

7. The medical device or part of a medical device as claimed in claim 1, wherein the thermoplastic composition contains at least 95% by weight of aromatic polycarbonate.

8. The medical device or part of a medical device as claimed in claim 1, wherein the thermoplastic composition consists of the following components:
A) at least 95% by weight of aromatic polycarbonate,
B) 0.2% to 1.5% by weight of functionalized siloxane polymer composed only of siloxane units as monomer units,
and
C) optionally one or more further additives selected from the group consisting of demolding agents, heat stabilizers, antioxidants, colorants, pigments.

9. The medical device or part of a medical device as claimed in claim 1, wherein 0.5% to 1% by weight of component B are employed.

10. The medical device or part of a medical device as claimed in claim 1, wherein the aromatic polycarbonate has a melt volume flow rate of 16 to 20 cm³/(10 min), determined according to ISO 1133:2012-03, at a test temperature of 300° C. and a load of 1.2 kg.

11. The medical device or part of a medical device as claimed in claim 1, wherein the functionalized siloxane polymer has a weight-average molecular weight M_{w}, determined by GPC in tetrahydrofuran (THF) calibrated against polystyrene standards, of >500 000 g/mol.

12. The medical device or part of a medical device as claimed in claim 1, wherein the functionalized siloxane polymer comprises dimethylsiloxane units.

13. The medical device or part of a medical device as claimed in claim 1, wherein the functionalized siloxane polymer comprises hydroxyl groups.

14. The use of functionalized siloxane polymer for improving the intralipid resistance of compositions based on aromatic polycarbonate.

15. The use as claimed in claim 14, wherein the functionalized siloxane polymer has a weight-average molecular weight M_{w}, determined by GPC in tetrahydrofuran (THF) calibrated against polystyrene standards, of >500 000 g/mol and comprises dimethylsiloxane units and hydroxyl groups.
